(19) United States
(12) Patent Application Publication
(10) Pub. No.: US 2026/0080592 A1
(43) Pub. Date: Mar. 19, 2026

(54) METHODS AND SYSTEMS FOR PROVIDING A REPRESENTATION OF A WHOLE SLIDE IMAGE

(71) Applicant: Siemens Healthineers AG, Forchheim (DE)

(72) Inventors: Svenja LIPPOK, Uttenreuth (DE); Gerardo HERMOSILLO VALADEZ, West Chester, PA (US)

(73) Assignee: Siemens Healthineers AG, Forchheim (DE)

(21) Appl. No.: 19/332,616

(22) Filed: Sep. 18, 2025

(30) Foreign Application Priority Data
Sep. 19, 2024 (DE) 10 2024 208 987.7

(51) Int. Cl. G06T 11/60; G06T 3/40; G06T 7/33; G16H 40/20

(52) U.S. Cl. CPC G06T 11/60; G06T 3/40; G06T 7/337; G16H 40/20; G06T 2200/24; G06T 2207/10056; G06T 2207/30024; G06T 2210/41

(57) ABSTRACT

A computer-implemented method, comprises: obtaining a case identifier indicating a digital pathology case to be reviewed by a user in a corresponding diagnostic task of the user on a user interface, the digital pathology case being associated with at least one whole slide image stored in an image database; determining conditional information applicable to the corresponding diagnostic task based on the case identifier; generating a representation of the at least one whole slide image for displaying, on the user interface, by processing the at least one whole slide image according to the conditional information; and providing the representation for displaying on the user interface.

### CROSS-REFERENCE TO RELATED APPLICATION(S)

The present application claims priority under 35 U.S. C. § 119 to German Patent Application No. 10 2024 208 987.7, filed Sep. 19, 2024, the entire contents of which is incorporated herein by reference.

### FIELD

One or more example embodiments of the present invention relate to methods and systems for determining and displaying a representation of a whole slide image. In particular, one or more example embodiments of the present invention relate to systems and methods for image processing of whole slide images, in particular, for deriving a medical diagnosis. Further, one or more example embodiments of the present invention relate to methods and systems for controlling a reading and reporting workstation in a digital pathology reading and reporting workflow.

### BACKGROUND

Reading and reporting of pathology images is performed in over 90% of all cancer cases with the final diagnosis of the cancer patient done by a pathologist. Despite of this importance, the process of digitizing the workflow has only just begun. Current reading and reporting platforms are not well suited for this growing amount of information and lack the focus on efficiency increases and workflow improvements that can be achieved by applying digital tools. One issue is that a pathologist has to manually select relevant image series from the available images of a patient (as done in the manual workflow when using the microscope), arrange the images in her or his viewer, apply the correct viewing parameters, call relevant additional information and display this information in a corresponding window in the viewer, select the correct image processing tools, and so forth.

Thereby the correct image series, the region of interest within the image, supplementary data to be shown, display settings, e.g., zoom levels, and so forth depend on various influencing factors which might change from patient to patient. One crucial parameter in this regard is the kind of histopathological stainings used for the images. In digital pathology, different stainings might bear very different implications in the reporting workflow and might, therefore, require displaying different pieces of information and different arrangements of the individual windows in a viewer.

Thus, there is a need for a system that can better support a user in this regard and leverage possibilities of pre-processing that reading and reporting with a microscope could not support. The system should be capable of automatically loading the relevant patient data and employ a suited layout in the corresponding viewing application.

### SUMMARY

It is an object of one or more embodiments of the present invention to provide systems and methods which render the reading and reporting workflow in digital pathology more efficient. In particular, it is an object of one or more embodiments of the present invention to improve the handling and processing of whole slide images aiming at obtaining a medical diagnosis based on the whole image.

At least this object is solved by a method for displaying a representation of a whole slide image, a system for displaying a representation of a whole slide image, corresponding computer-program products, and corresponding computer-readable storage media according to the main claims. Alternative and/or preferred embodiments are object of the dependent claims and/or the aspects as herein described.

In the following, a technical solution according to one or more example embodiments of the present invention is described with respect to the claimed apparatuses as well as with respect to the claimed methods. Features, advantages, or alternative embodiments described herein can likewise be assigned to other claimed objects and vice versa. In other words, claims addressing the inventive method can be improved by features described or claimed with respect to the apparatuses. In this case, e.g., functional features of the method are embodied by objective units or elements of the apparatus.

The technical solution will be described both with regard to methods and systems for providing a representation of a whole slide image and also with regard to methods and systems for providing a trained function configured for extracting conditional information from the whole slide image and/or corresponding non-image data, wherein the conditional information serves as indication for the generation of the representation. Features and alternate forms of embodiments of data structures and/or functions for methods and systems for providing trained functions can be transferred to analogous data structures and/or functions for methods and systems for providing the image acquisition information. Analogous data structures can, in particular, be identified by using the prefix “training”. Furthermore, the prediction functions used in methods and system for providing information can, in particular, have been adjusted and/or trained and/or provided by methods and systems for adjustment of trained functions.

According to an aspect, a computer-implemented method for displaying a representation of a whole slide image is provided. The method comprises a plurality of steps. One step is directed to obtain a case identifier indicating a digital pathology case to be reviewed by a user in a corresponding diagnostic task of the user in a user interface of a user end device, the digital pathology case being associated with at least one whole slide image stored in an image database. Another step is directed to determine a conditional information applicable to the diagnostic task using (based on) the case identifier. Another step is directed to generate a representation of the whole slide image for displaying in a user interface by processing the whole slide image according to the conditional information. Another step is directed to prompt a displaying of the representation in the user interface.

Whole-slide images may be two-dimensional digital images having a plurality of pixels. Whole slide images may have a size of at least 4.000×4.000 pixels, or at least 10.000×10.000 pixels, or at least 1E6×1E6 pixels.

A whole-slide image may image a tissue slice or slide of a patient. The preparation of the tissue slices from the tissue samples can comprise the preparation of a section from the tissue sample (for example with a punch tool), with the section being cut into micrometer-thick slices, the tissue slices. Another word for section is block or punch biopsy. Under microscopic observation, a tissue slice can show the fine tissue structure of the tissue sample and, in particular, the cell structure or the cells contained in the tissue sample. When observed on a greater length scale, a whole-slide image can show an overview of the tissue structure and tissue density. The tissue may have been taken from a tumor the patient is suffering from. In particular, the tissue may show a manifestation of a cancerous disease of the patient, such cells of a tumor.

The preparation of a tissue slice further may comprise the staining of the tissue slice with a histopathological staining. The staining in this case can serve to highlight different structures in the tissue slice, such as, e.g., cell walls or cell nuclei, or to test a medical indication, such as, e.g., a cell proliferation level. Different histopathological stains are used for different purposes in such cases.

To create the whole-slide image, the stained tissue slices are digitized or scanned. To this end, the tissue slices are scanned with a suitable digitizing station, such as, for example, a whole-slide scanner, which preferably scans the entire tissue slice mounted on an object carrier and converts it into a pixel image. In order to preserve the color effect from the histopathological staining, the pixel images are preferably color pixel images. Since in the prediction both the overall impression of the tissue and also the finely resolved cell structure may be of significance, the whole slide images typically have a very high pixel resolution. The data size of an individual image can typically amount to several gigabytes.

The whole slide image may be stored in a standard image format such as the Digital Imaging and Communications in Medicine (DICOM) format and in a memory or computer storage system such as a Picture Archiving and Communication System (PACS), a Radiology Information System (RIS), and the like. Whenever DICOM is mentioned herein, it shall be understood that this refers to the “Digital Imaging and Communications in Medicine” (DICOM) standard, for example according to the DICOM PS3.1 2020c standard (or any later or earlier version of said standard).

The representation may be generated by processing the whole slide image wherein the processing depends on the conditional information.

The representation may comprise one or more two-dimensional representation images rendered from the whole slide image. The representation images may comprise a plurality of image pixels. In particular, the representation images may be two-dimensional renderings of the whole slide image or of different views of the medical image, in particular of different regions (of interest) of the whole slide image. The representation image(s) may have a resolution which is adapted to the resolution of the user interface of the workstation, e.g., 1920×1080 or 4096×2160 pixel.

Renderings may, in general, rely on known rendering procedures. Thereby, the views, regions, and the rendering may depend on the image conditional information. For instance, the conditional information may suggest a certain rendering or pre-processing and/or a particular view, zoom level, etc.

Further, the representation may comprise a graphical user interface in which the representation images are included at a predefined position. In particular, the graphical user interface may be specifically configured to derive a certain medical diagnosis based on the whole slide image. Via the graphical user interface, the user may inspect the whole slide image, make measurements and record a medical diagnosis (e.g., in the form of a medical report). Further, the graphical user interface may be configured such that the user may control the user interface and/or the reading and reporting workplace/workstation based on inputs in the graphical user interface.

A case identifier may comprise an electronic identifier configured to identify the digital pathology case in a healthcare information system. For instance, the electronic identifier may comprise a patient name, a patient identification number, a case identification number and the like.

The digital pathology case may represent the entirety of information associated to a patient's case in the healthcare information system comprising information and data directly associated to the patient, such as the whole slide images of the patient or an electronic medical record of the patient, and data and information not directly associated to the patient but of general relevance for the case, such as medical or clinical guidelines, internal workflows, preferences of the user and the like.

The diagnostic task may comprise the specific question the user has to answer in a diagnostic workflow. Further, the diagnostic task may comprise the artefact, the user has to provide such as the medical report to be provided. Further, the diagnostic task may comprise an indication of the examination the user has to review.

The conditional information may comprise circumstances or boundary conditions for the diagnostic task and/or for the reviewing of the digital pathology case.

According to some examples, the conditional information comprises an image acquisition information and/or an information about the diagnostic task.

According to some examples, the image acquisition information comprises at least one of: a property of the at least one whole slide image, a staining type of the at least one whole slide image, a location of the at least one whole slide image in a biopsy block, a number of whole slide images associated with the digital pathology case, and/or an image quality of the at least one whole slide image.

According to some examples, the information about the diagnostic task comprises at least one of a type of the diagnostic task to be performed, a suspected diagnosis, an intended recipient of a diagnosis determined based on the diagnostic task.

Determining the conditional information using the case identifier may comprise querying the healthcare information system/the database based on the case identifier and determining the conditional information based on the query result.

Generating the representation may comprise obtaining and/or processing the whole slide image according to the conditional information. For instance, this may comprise obtaining, in particular, selecting, the whole slide image based on the information about the diagnostic task and/or processing the whole slide image bases on the image acquisition information or vice versa. Obtaining may comprise loading the whole slide image from the database.

The user interface may be adapted to interface with one or more users of the system, e.g., by displaying the result of the processing of the computing unit to the user (e.g., in a graphical user interface) or by allowing the user to make inputs for arriving at a medical diagnosis.

The database may be realized as a cloud storage or as a local or spread storage. The database may be comprised in a healthcare information system. The healthcare information system may be configured to acquire, store and/or forward at least whole slide images and, optionally, also other data related to the digital pathology case. According to some examples, the healthcare information system comprises one or more databases for storing whole slide images and/or supplementary information. Further, the healthcare information system may comprise one or more imaging modalities, such as a slide scanning apparatus or radiology imaging modalities or the like.

The user end device may be a desktop PC or laptop or tablet. The user interface may comprise one or more displays and input devices such as touch screens, keyboards, microphones, computer mouses and the like. The user end device may be in data communication with the database. According to other examples, the user end device may be in data communication with server in the healthcare information system which is, in turn, in data exchange with the database.

By automatically generating the representation using the conditional information, views which will likely be required based on the circumstances of the case are automatically generated and offered to a user. This relieves the user from the routine but tedious task of setting up the representation herself for the further diagnosis of the whole slide image.

According to an aspect, a computer-implemented method for controlling a workstation in a digital pathology reading and reporting workflow comprising reviewing a digital pathology case according to a diagnostic task is provided. The method comprises a plurality of steps. One step is directed to obtain a case identifier indicating the digital pathology case, the digital pathology case being associated with at least one whole slide image stored in an image database in communication with the workstation. Another step is directed to determine a conditional information applicable to the diagnostic task using (based on) the case identifier. Another step is directed to provide control signal suited to generate and display a representation of the whole slide image for displaying in a user interface by processing the whole slide image according to the conditional information. Another step is directed to prompt a displaying of the representation in the user interface by inputting the control signal in the workstation.

According to some examples, a plurality of different types of conditional information may be preconfigured and the step of determining the conditional information may comprise classifying the digital pathology case according to the plurality of different types. Thereby, the step of classifying may comprise accessing data, in particular comprising the at least one whole slide image, associated with the digital pathology case using the case identifier and classifying the digital pathology case based on the accessed data.

According to an aspect, the step of determining the conditional information comprises obtaining non-image information associated with the case identifier from a database different than the image database, and determining the conditional information based on the non-image information.

According to some examples, the non-image information comprises structured and/or unstructured natural language text.

According to some examples, the supplementary information comprises one or more of the following elements:
a prior medical report of the patient, and/or
a-priori knowledge of at least one type of medical problem the patient is suspected to have, and/or
an indication of the diagnostic task to be performed, e.g., in case of a resection based on diagnosis of a biopsy of the same tumor, and/or
an electronic health record of the patient, which may, e.g., comprise lab data like a PSA value as a tumor marker.

According to some examples, the a-priori knowledge may be obtained via a referral information associated with the patient, via information obtained based on a preliminary result from initial stainings ordered for the case, or via frozen section analysis results.

Obtaining the non-image information may comprise querying a healthcare information system and/or corresponding non-image databases such as a HIS (hospital information system), a LIS (laboratory information system), an EMR-system (electronic medical record system) and the like for supplementary information of the patient. Such supplementary information may be obtained in the form of one or more EMR-files (electronic medical record-files), for instance. Further, querying healthcare information systems may be based on a patient identifier such as an ID or the patient's name, electronically identifying the patient in the system.

Leveraging the additionally available information of the digital pathology case may give additional insights in the conditional information and/or can be used to safeguard a prediction made, e.g., based on the whole slide images.

According to some examples, the step of obtaining the non-image information comprises accessing a meta-data structure of the whole slide images, in particular, a header, further in particular, a DICOM header, and the step of determining the conditional information comprises searching the meta-data for conditional information.

According to some examples, the method comprises providing a generative AI function configured to predict conditional information for diagnostic tasks based on non-image information, and the step of determining the conditional information comprises applying the generative AI function to the non-image information. According to some examples, the generative AI function may be based on a large language model.

A large language model (LLM) is a language model characterized by its large size. In particular, the large language model may be based on a transformer architecture. According to some examples, the large language model may comprise or may be based on models available at the date of filing such as GPT models (e.g., GPT-3.5 and GPT-4), PaLM, LLaMa, LLaMa 2, Falcon, Whisper, and the like.

In particular, the large language may comprise an off-the-shelf large language model which is used as is. According to other examples, the large language model may be further trained based on the domain knowledge of the healthcare information systems. In this regard, the large language model may be provided with examples of relevant conditional information for example digital pathology cases.

According to some examples, the method further comprises determining the conditional information based on the non-image information before loading (any) whole slide image associated to the digital pathology case from the database into the workstation. This may have the advantage that the traffic in the healthcare information system may be reduced as the notoriously huge whole slide image are only loaded if needed.

According to an aspect, the step of determining the conditional information comprises providing a machine-learned function configured to determine a conditional information based on image data comprised in whole slide images and applying the machine-learned function to the at least one whole slide image.

According to some examples, the step of determining the conditional information further comprises obtaining the at least one whole slide image from the image database using the case identifier.

According to some examples, this may comprise querying the image database for the at least one whole slide image using the case identifier.

Leveraging the information comprised in the image data of the whole slide images is fail-safe as this information is always present. By using a trained function, a scheme is provided which is able to adapt to new circumstances and to detect and extrapolate patterns, e.g., stemming from different slide scanners, different manufacturers of stains, different slide preparations and the like.

The trained function is configured (or has been trained) to extract conditional information directly from whole slide images. The conditional information thus extracted may, in particular, relate to image acquisition information. Applying the trained function may comprise providing the trained function and/or inputting the respective data in the trained function.

In general, a trained function mimics cognitive functions that humans associate with other human minds. Other terms for machine-learned function, may be machine-learned function, trained machine learning model, trained mapping specification, mapping specification with trained parameters, function with trained parameters, algorithm based on artificial intelligence, or machine learned algorithm.

In general, parameters of a machine-learned function can be adapted via training. In particular, supervised training, semi-supervised training, unsupervised training, reinforcement learning and/or active learning can be used. Furthermore, representation learning (an alternative term is “feature learning”) can be used. In particular, the parameters of the machine-learned function can be adapted iteratively by several steps of training.

In particular, a trained function can comprise a neural network, a support vector machine, a decision tree and/or a Bayesian network, and/or the trained function can be based on k-means clustering, Q-learning, genetic algorithms and/or association rules. In particular, a neural network can be a deep neural network, a convolutional neural network or a convolutional deep neural network. Furthermore, a neural network can be an adversarial network, a deep adversarial network and/or a generative adversarial network.

The trained function may be generally configured to determine conditional information based on image data obtained from whole slide images. For instance, the trained function may be configured to extract one or more features from the image and map/classify these features into a feature space associated with different conditional information. Thus, the trained function may comprise a feature extractor and a classifier. In particular, the feature extractor and the classifier may be implemented as a neural network, in particular, a convolutional neural network, with some network layers trained to extract features and other network layers being trained to provide a classification according to the most likely image acquisition information.

In principle, all suited trained functions may be used for obtaining the conditional information from the image data.

According to some examples, however, the trained function comprises at least one of: a convolutional neural network, a transformer network, and/or a FocalNet.

A convolutional neural network is a neural network that uses a convolution operation instead of general matrix multiplication in at least one of its layers (so-called “convolutional layer”). In particular, a convolutional layer performs a dot product of one or more convolution kernels with the convolutional layer's input data/image, wherein the entries of the one or more convolution kernel are the parameters or weights that are adapted by training. In particular, one can use the Frobenius inner product and the ReLU activation function. A convolutional neural network can comprise additional layers, e.g., pooling layers, fully connected layers, and normalization layers.

For an example for the general usability of convolutional neural networks for deriving conditional information, reference is made to van der Voort et al., DeepDicomSort: An Automatic Sorting Algorithm for Brain Magnetic Resonance Imaging Data, in Neuroinformatics, 2021, January, 19(1):159-184, doi: 10.1007/s12021-020-09475-7, the contents of which are herein included by reference in their entirety. While being applied on radiology image data in van der Voort et al., the inventors have recognized that the architecture may, in principle, also be applied to whole slide images.

By using convolutional neural networks, input whole slide image data can be processed in a very efficient way, because a convolution operation based on different kernels can extract various image features, so that, by adapting the weights of the convolution kernel, the relevant frequency patterns can be readily found during training. Furthermore, based on the weight-sharing in the convolutional kernels, less parameters need to be trained, which prevents overfitting in the training phase and allows to have faster training or more layers in the network, improving the performance of the network.

A transformer network is a neural network architecture generally comprising an encoder, a decoder, or both an encoder and decoder.

In some instances, the encoders and/or decoders are composed of several corresponding encoding layers and decoding layers, respectively. Within each encoding and decoding layer is an attention mechanism. The attention mechanism, sometimes called self-attention, relates data elements (such as words or pixels) within a series of data elements to other data elements within the series.

The encoder, in particular, may be configured to transform the input whole slide image into a numerical representation. The numerical representation may comprise a vector per input token (e.g., per image patch). The encoder may be configured to implement an attention mechanism so that each patch is affected by the other patches in the input. In particular, the encoder may be configured such that the representations resolve the desired output, i.e., the image acquisition information of the trained function.

The decoder, in particular, may be configured to transform an input into a sequence of output tokens. In particular, the decoder may be configured to implement a masked self-attention mechanism so that each vector of a token is affected only by the other tokens to one side of a sequence. Further, the decoder may be auto-regressive in the sense that intermediate results (such as a previously predicted sequence of tokens) are fed back.

According to some examples, the output of the encoder is input into the decoder.

Further, the transformer network may comprise a classification module or unit configured to map the output of the encoder or decoder to a set of learned outputs in the form of the image acquisition information.

In particular, the transformer network may be embodied as a vision transformer. The vision transformer may be configured to break down input image data into patches and tokenize them (extracting representation vectors), before applying the tokens to a standard transformer architecture. The vision transformer may comprise an attention mechanism configured to repeatedly transform representation vectors of image patches for increasingly incorporating semantic relations between image patches. In this regard, the inventors have recognized that the usage of vision transformers enabling a patch-wise processing is particularly advantageous in connection with notoriously large whole slide images.

According to some examples, the vision transformer may be obtained by training a masked autoencoder. A masked auto-encoder comprises two vision transformers put end-to-end. The first one takes in whole slide image patches with positional encodings and outputs vectors representing each patch. The second one takes in vectors with positional encodings and outputs image patches again. During training, both vision transformers are used. During use, the first vision transformer may be used as encoder and/or the second vision transformer may be used as generative AI function.

For a review on transformer networks, reference is made to Vaswani et al., “Attention Is All You Need”, in arXiv: 1706.03762, Jun. 12, 2017, the contents of which are herein included by reference in their entirety.

An advantage of transformer networks is that, due to the attention mechanism, transformer networks can efficiently deal with long-range dependencies in input data. Further, encoders used in transformer networks are capable of processing data in parallel which saves computing resources in inference. Moreover, decoders of transformer networks, due the auto-regression, are able to iteratively generate a sequence of output tokens with great confidence.

FocalNet is short for focal modulation network. A FocalNet is a neural network that uses a focusing mechanism to enable the model's interaction with the input, in this case the whole slide image. Specifically, FocalNets use a lightweight element-wise multiplication as a focusing operator to see or interact with the input with the proposed modulator. The modulator is computed with a focal aggregation procedure in two steps: focal contextualization to extract contexts from local to global regions at different granularity levels and gated aggregation to condense all context features at different granularity levels into the modulator. For a review on FocalNets, reference is made to Yang et al., Focal Modulation Networks, arXiv:2203.11926, the contents of which are herein included by reference in their entirety.

The inventors have recognized that FocalNet may outperform attention-based neural networks when dealing with large whole slide images.

According to some examples, the conditional information comprises at least one tumor cell detected in the at least one whole slide image and the machine-learned function is configured to detect tumor cells in whole slide images (or discriminate tumor cells from healthy cells).

Recognizing tumor cells may have the advantage that the presence of tumor cells may imply different kinds of representations as compared to a case where no tumor cells are present.

According to an aspect, the machine-learned function is configured to determine histopathological stainings of whole slide images, and the conditional information comprises a staining type of the at least one whole slide image.

As the histopathological staining used may be characteristic for the underlying clinical task as well as for the subsequent automated preparation of the case for a user, extracting this information from the whole slide images enables to efficiently automate the further processing of the whole slide image and the configuration of the workplace.

According to an aspect, the step of determining the conditional information further comprises combining the conditional information based on the non-image information with the conditional information based on the image data so as to provide the conditional information.

By combining the two sources, orthogonal information may be used. This is beneficial for the reliability of the method.

According to an aspect, the step of determining the conditional information may comprise comparing/verifying the conditional information obtained based on image data with the image acquisition information obtained based on non-image data, wherein, in the step of providing the conditional information, the conditional information is provided additionally based on the step of comparing/verifying. Specifically, the step of comparing/verifying may comprise determining if the image acquisition information sufficiently correspond.

According to an aspect, the step of determining the conditional information may comprise determining a confidence value of the conditional information obtained based on non-image data (or, as the case may be, of the conditional information obtained based on image data), comparing the confidence value to a predetermined threshold, determining the conditional information based on image data (or, as the case may be, of the conditional information obtained based on non-image data) if the confidence value is below the predetermined threshold, and determining the conditional information based on the conditional information based on image data (or, as the case may be, of the conditional information obtained based on non-image data).

With that, a double processing can be avoided. Thereby, it may be preferred according to some examples to first calculate the conditional information based on non-image information and only execute the computationally more expensive determination based on image data if needed.

A confidence value (or confidence measure, or confidence level, or certainty score, or trust level) may indicate how confident the prediction of the conditional information is. A certainty measure, according to some examples, may comprise a numerical value or a collection of numerical values (e.g., a vector) that indicate, according to a model or algorithm, the degree of certainty or uncertainty a conditional information or part of a conditional information is afflicted with and/or, generally, the quality of a certain conditional information. According to some examples, obtaining the certainty measures may comprise assigning a value/certainty measure to each element of the conditional information.

According to some examples, a certainty measure may be provided for the treatment response prediction as whole. According to other examples, a certainty measure may be provided per element of the treatment response prediction, in particular, per treatment option comprised in the treatment response prediction.

Confidence values may be calculated using certainty calculation modules which may be integrated in the trained functions or provided as separate modules, essentially as described in DE 10 2023 209 304 A1, the contents of which are incorporated herein in its entirety.

According to some examples, the non-image data comprises laboratory data, the laboratory data in particular comprising a tumor marker. According to some examples, the laboratory data may indicate a certain organ and the conditional information based on the non-image data may comprise the indication of the certain organ. In particular, the laboratory data may indicate the certain organ by virtue of being specific for the certain organ. In turn, determining the conditional information may comprise identifying the certain organ the laboratory data is specific for.

For example, the laboratory data may comprise a PSA value which is specific for the prostate as the certain organ.

According to an aspect, the digital pathology case is associated with a plurality of different whole slide images, and the step of generating the representation comprises selecting the at least one whole slide image from the plurality of different whole slide images based on the conditional information.

With that, the whole slide image which is suited for the representation may be automatically identified. This may spare the user from manually going through the available whole slide images and open these one after the other until a suitable image was found. The suited whole slide image in this regard may be one with the correct stain taken from a suitable biopsy block and section position.

According to some examples, the conditional information indicates a certain organ and the whole slide image may be selected so as to represent the certain organ. For instance, whole slide images may be selected which we prepared from biopsies of the certain organ.

According to some examples, the conditional information may comprise the task of grading a tumor type and the method further comprises providing a machine-learned function configured to recognize tumor cells of the tumor type, applying the machine-learned function at least a subset of the plurality of whole slide images so as to determine a number of tumor cells for the subset and selecting the at least one whole slide image comprises selecting from the subset based on the numbers of tumor cells, in particular, selecting the whole slide image with the highest number of tumor cells as the at least one whole slide image.

With that, a user can be automatically provided with those whole slide images which are best suited for the grading. As to the machine learned functions for detecting tumor cells, all in principle suitable functions may be used. For instance, reference is again made to EP 23 18 4424.

According to an aspect, the different whole slide images are stained with different histopathological stainings, and the step of generating the representation comprises determining, based on the conditional information, a desired histopathological stain, determining, among the plurality of whole slide images, a subgroup stained with the desired histopathological staining, and selecting the at least one whole slide image from the subgroup.

According to some examples, the selection from the subgroup may comprise a further automated selection based on the conditional information, in particular of the image acquisition information of the whole slide images in the subgroup. According to some examples, a selection based on image quality may be made.

By automatically selecting appropriate stains, the user may automatically be provided with suitable whole slide images for the further diagnosis. For instance, if the conditional information indicates that a keratin expression level needs to be determined, the available whole slide images may be searched for an image with a IHC stain comprising biomarkers (e.g., in the form of antibodies) configured to target (cytoskeletal) keratins.

According to an aspect, the step of determining the conditional information comprises determining an image acquisition information for the plurality of whole slide images as herein described (in particular, using the trained function) and determining the subgroup and/or selecting from the subgroup based on the image acquisition information.

According to some examples, the conditional information comprises an indication of an organ of interest (also referred to as “certain organ”) and the desired histopathological staining is determined according to the indication of the organ of interest. Specifically, a histopathological staining may be selected which is specific and/or specifically binds to the cells of the organ of interest.

For instance, if laboratory data comprises a PSA value, the organ of interest might be determined to be the prostate. That followed, whole slide images may be selected which have been stained using prostate specific antigens as indicator of the prostate as tumor site of origin.

According to an aspect, the step of generating the representation comprises determining a displaying setting for generating the representation based on the conditional information and processing the at least one whole slide image according to the determined displaying setting, the displaying setting being selected from: a contrast setting, a brightness, an intensity windowing, an image enhancement, a look-up table, a viewing plane, a segmentation mask, an order of the at least one whole slide images within other images, and/or a zoom level.

By automatically, determining displaying settings, the user is automatically provided with appropriate parameters and does not have to set these herself. Accordingly, the workflow may be further automized and the user is relieved.

According to some examples, the displaying setting may comprise settings for a plurality of different whole slide images with defined, different individual displaying settings per whole slide image. According to some examples, the different individual displaying settings may comprise instructions for a representation displaying, e.g., the same region of interest of one whole slide image at different zoom levels or displaying corresponding regions of interest of multiple whole slide images from the same block.

According to some examples, the displaying setting may comprise a display order for multiple whole slid images (in particular the at least one whole slide image and the comparative whole slide images). In particular, consecutive whole slide images or complementary stainings may be displayed next to each other.

According to some examples, the conditional information comprises a staining type of the at least one whole slide image and determining the displaying setting comprises selecting a zoom level according to the staining type. Further, selecting the zoom level may comprise selecting the zoom level from a plurality of predetermined different zoom levels wherein, optionally, the plurality of different zoom levels at least comprise a cellular zoom level and a tissue zoom level.

Thereby the cellular zoom level may be configured such that individual cells may be resolved by a user in the representation. This may mean that only a part of the whole slide image comprising a limited number of cells, e.g., tens to hundreds of cells, is shown. The tissue zoom level may be configured such that entire tissue or tumor regions may be inspected where individual cell cannot be readily resolved by a user in the representation. According to some examples, the tissue zoom level may be such that the entire whole slide image is shown. Naturally, there are further zoom levels possible, e.g., a cell-nuclei-zoom level which is greater than the cellular zoom level and which may be configured such that cell nuclei can be resolved in the representation, or a blood vessel zoom level which is lower that the cellular zoom level but greater than the tissue zoom level and may be configured such that blood vessels can be readily resolved.

This has the advantage that different stainings visualize different objects with different sizes that need to be studied at different magnifications, e.g., cell nuclei, RNA cluster, blood vessels, tumor regions. By automatically selecting an appropriate zoom level, a user is provided with additional assistance.

According to an aspect, the conditional information comprises a region of interest in the at least one whole slide image, the step of generating the representation comprises processing the whole slide image so as to crop the whole slide image according to the region of interest.

The automated zooming-in on the region of interest is advantageous as whole slide images are huge and scrolling and zooming through a whole slide image may take a considerable amount of time. This is important because whole slide images generally show highly heterogeneous tissue and not all regions are equally relevant for diagnosis.

According to other examples, the conditional information comprises a staining type of at least one whole slide image and the region of interest is determined based on the staining type. This is advantageous because different stainings generally highlight different areas of interest, e.g., tumor and tumor microenvironment.

According to some examples, the step of determining the conditional information comprises providing a computer-aided detection function configured to detect medical findings in whole slide images, applying the computer aided detection function to the whole slide image so as to detect one or more findings, and determining the region of interest based on the medical findings.

For instance, the computer-aided detection function may be configured to identify certain tissue types in whole slide images such as cancerous growths, necrotic tissue, and the like. In principle, there exists a plurality of different detection algorithms which might be used in this regard. For instance, reference is made to EP 23 18 4424, the contents of which are incorporated herein in their entirety by reference.

According to some examples, the step of determining the conditional information comprises providing a computer-aided detection function configured to detect existing annotations in whole slide images, applying the computer aided detection function to the whole slide image so as to detect one or more existing annotations, and determining the region of interest based on the existing annotations.

Existing annotations may relate to markings and comments which have been added to a whole slide image, e.g., in a previous examination. To detect existing annotations, the detection function may be configured to detect text characters or markings in image data. According to some examples, vision transformers may be used in this regard.

According to some examples, the method further comprises retrieving, from the image database and based on the conditional information, a comparative whole slide image, processing the comparative whole slide image so as to generate a comparative representation for displaying in the user interface, and displaying the comparative representation in the user interface.

According to some examples, the comparative whole slide image may have the same or similar image acquisition information as the at least one whole image.

According to some examples, the comparative whole slide image may be selected from a case for which the same stainings are available and/or a whole slide image from a former case of the same patient to identify metastasis or recurrence of a tumor.

The comparative whole slide image may relate to the same patient as the at least one whole slide image. According to some examples, the at least one whole slide image was acquired from the patient at a first point in time and the comparative medical image was acquired from the patient at a second point in time different than the first point in time. The comparative whole slide image may relate to a different patient as the at least one whole slide image. In particular, the comparative whole slide image may have a degree of similarity to the whole slide image. The comparative whole slide image may be obtained from a database of comparative medical images. Further, the comparative whole slide image may be obtained from an electronic medical textbook. The comparative whole slide image may be associated with a verified medical diagnosis.

By offering a comparative medical image to a user, the user can be provided with additional information for deriving a medical diagnosis.

According to an aspect, generating the comparative representation comprises processing the comparative whole slide image in an analogous way as the at least one whole slide image, and/or calculating and image registration between the at least one whole slide image and the comparative whole slide image and processing the comparative whole slide image using the image registration (so that the comparative whole slide image shows essentially the same image section as the at least one whole slide image).

A registration may comprise a mathematical transformation from the image space of the at least one whole slide image to the image space of the comparative whole slide image. With that, corresponding regions in the whole slide images may be identified, enabling, for instance, to display corresponding regions of interest.

According to some examples, the registration may comprise a rigid registration. A rigid registration may comprise a registration in which the coordinates of pixels in one image are subject to rotation and translation in order to register the image to another image. According to some examples, the registration may comprise and affine registration. An affine registration may comprise a registration in which the coordinates of data points in one image are subject to rotation, translation, scaling and/or shearing in order to register the image to another image. Thus, a rigid registration may be considered to be a particular type of affine registration. According to some examples, the registration may comprise a non-rigid registration. A non-rigid registration may provide different displacements for each pixel of the image to be registered and can, for example, use non-linear transformations, in which the coordinates of pixels in one image are subject to flexible deformations in order to register the image to another image. Non-linear transformations may, according to some examples, be defined using vector fields such as warp fields, or other fields or functions, defining an individual displacement for each pixel/voxel in an image. For more detailed information about image registration, reference is made to US 2011/0 081 066 and US 2012/0 235 679. Rigid image registration is very effective in cases when no deformations are expected. In comparison to rigid image registration, non-rigid image registration has a significantly greater flexibility as non-rigid image registrations can manage local distortions between two image sets but can be more complex to handle.

Using image registration techniques has the advantage that the whole slide images can be transformed into a common coordinate system. With that, it can be ensured that, e.g., regions of interest have the same scale and the same location. In turn, processing results can be more readily compared.

By using an analogous processing (which may mean that the same image processing steps are applied to the comparative whole slide image as to the at least one whole slide image), the comparative whole slide image can be more readily compared to the whole slide image and/or the same measurements may be made.

According to an aspect the at least one whole slide image was prepared from a section of a biopsy block, the conditional information comprises a location of the section in the biopsy block, the comparative whole slide image is selected based on the location, in particular, based on a proximity to the location.

By basing the selection of the comparative whole slide image on the location of the at least on whole slide image, whole slide images may be selected which are proximal or adjacent to one another. This is beneficial because proximal or neighboring sections are morphologically similar and may be more readily compared, in particular, if the two whole slide images have been stained differently.

According to some examples, the selection of the comparative whole slide image comprises determining a morphological similarity between the at least one whole slide image and the comparative whole slide image and selecting the comparative whole slide image based on the morphological similarity.

With that, whole slide images may be deliberately selected based on how well they can be compared with the at least one whole slide image. This may ensure that the user is not bothered with dissimilar whole slide images. Of note, this may even be relevant for neighboring whole slide images differences in the image quality, the preparation, or the sectioning might still render neighboring whole slide images incomparable.

According to an aspect, the method further comprises selecting, based on the image condition information, a displaying protocol including a rule set for displaying one or more representations of whole slide images in a user interface, wherein, in the step of displaying, the representation is displayed based on the displaying protocol.

Current reading and reporting systems use general techniques known as “displaying or hanging protocols” to format the display or layout of whole slide images or excerpts from whole slide images. Such protocols allow a user to specifically set displaying environments according to staining, anatomy, and procedure. Displaying protocols present one or more perspectives or views (e.g., in the form of the aforementioned representations) of the whole slide image to the user. Representations may be grouped and located in a graphical user interface. In addition, displaying protocols may comprise rules or instructions for obtaining additional information such as comparative medical images acquired before or after the whole slide image or for applying certain image analysis tools or existing medical reports or reporting templates.

According to some examples, the displaying protocol may be selected from a plurality of predefined displaying protocols. The predefined displaying protocols may be configured according to different use cases and the step of selecting may comprise identifying a use case based on the conditional information and selecting the displaying protocol corresponding to the identified use case.

By selecting the appropriate displaying protocol, the user is automatically provided with a user interface specifically adapted for the image data and the diagnostic task. This not only relieves the user but also automates the image processing towards the provision of a medical diagnosis.

According to some examples, the method comprises retrieving a (existing) medical report of the patient from a report data base using the case identifier and including the medical report in the representation.

According to an aspect, the method further comprises selecting, based on the conditional information, in particular, a staining type of the at least one whole slide image, an image processing tool configured to provide an image processing result, apply the selected image processing tool to the at least one whole slide image so as to generate the image processing result, and displaying the image processing result in the user interface.

According to some examples, the image processing tools may also be applied to any comparative whole slide image.

The image processing tool may be selected from a plurality of available image processing tools. The image processing tools may be specific to certain use cases/conditional information. For example, the plurality of image processing tools may comprise tools specific for a certain staining and/or diagnostic task.

Further, the image processing tool may be selected from one or more segmentation tools, one or more detection/classification tools, one or more cell counting tools etc. Accordingly, the image detection result may comprise: a detection result of a medical finding in the whole slide image, a classification of a tissue type in the whole slide image, a segmentation of the whole slide image, and/or a cell count.

According to some examples, the image processing tools may comprise one or more of tools configured to
detect or classify tumor cells, e.g., based on H&E stained cells,
predict response to therapies, e.g., based on a quantification of PD-L1 positive tumor cells,
measure microsatellite instability, e.g., based on MLH1 stained slides,
measure disease progression, e.g., based on Ki67 or FoxP3 classification,
quantify disease grading e.g., based on ER, PR, Her2 positive cell counting in breast cancer tissue,
measure the tumor site of origin e.g., based on quantifying staining of organ specific antigens, and/or
detect tumor invasiveness by, e.g., quantifying epidermal growth factor (EGFR).

According to some examples, the image processing result may be displayed in the representation, e.g., as an overlay over a rendering of the at least one whole slide image.

By automatically identifying and applying image processing tools, results can be automatically generated. The user is provided with cues for arriving at a medical diagnosis without having to search the library of available tools for appropriate ones.

According to an aspect, the method further comprises selecting, based on the conditional information, a reporting template for producing a medical report corresponding to the at least one whole slide image, and providing the reporting template via the user interface.

A reporting template may be a pre-configured data structure or building block or module on the basis of which a structured medical report may be generated. A medical report may be generated based on one or more reporting templates.

Selecting the reporting template may comprise a selection from a plurality of reporting templates. Each reporting template may be specific to a certain conditional information. For instance, a certain reporting template may be associated to a digital pathology case of liver biopsy, while another reporting template is associated to a biopsy of prostate.

Each reporting template may specify one or more data fields which have to be addressed or filled for completing the medical report. Further, a reporting template may comprise one or more pull-down menus with items a user can select. As such, a reporting template may also be conceived as an input form or mask structuring the information to be provided for a given diagnostic task.

According to some examples, the method may further comprise pre-filling the reporting template based on the conditional information and/or any other image processing results, e.g., as obtained by applying image processing tools.

By fetching appropriate reporting templates, the user is automatically provided with appropriate template data structures. In turn, the user is relieved from the burden of having to search for correct template data structure on her own in potentially vast databases.

According to an aspect, the method further comprises determining, based on the conditional information, a designated recipient of the whole slide image in a medical information system comprising a plurality of possible recipients, the possible recipients comprising one or more of a database, an image processing server, a worklist of a user, a reading and reporting workstation of a user, and/or a data exchange server for exchanging data to the outside of the healthcare information system. Optionally, the method may include forwarding/transmitting the whole slide image to the designated recipient.

By identifying designated recipients, the image may be automatically routed to the correct place in the healthcare information system.

According to an aspect, the method further comprises monitoring one or more prior actions of the user, and determining the conditional information based on the prior action.

The prior actions may be interactions of the user with the user interface, in particular, for generating prior representations.

The prior actions may be prior actions within the digital pathology case or general prior actions the user applied in other cases as well.

The prior actions may also be designated as “viewing behavior” and involve the selection of certain stainings, hanging orders, regions of interest, zoom levels and other viewing parameters.

With that, preferences and displaying parameters may be automatically transferred to new whole slide images of the case or new cases.

According to an aspect, a system for providing a representation of a whole slide image for displaying the representation in a user interface is provided. The system comprises a computing unit and an interface unit. The interface unit is in data communication with the user interface and an image database, wherein the user interface is configured to display information to a user, and the image database is configured to store whole slide images. The computing unit is configured to obtain a case identifier indicating a digital pathology case to be reviewed by a user in the user interface within a corresponding diagnostic task, the digital pathology case being associated with at least one whole slide image stored in the image database, determine a conditional information applicable to the diagnostic task based on the case identifier, generate a representation of the whole slide image for displaying in the user interface by processing the whole slide image according to the conditional information, and to provide the representation to the user interface.

The computing unit(s) may be realized as a data processing system or as a part of a data processing system. Such a data processing system can, for example, comprise a cloud-computing system, a computer network, a computer, a tablet computer, a smartphone and/or the like. The computing unit can comprise hardware and/or software. The hardware can comprise, for example, one or more processors, one or more memories and combinations thereof. The one or more memories may store instructions for carrying out the method steps according to embodiments of the present invention. The hardware can be configurable by the software and/or be operable by the software. Generally, all units, sub-units, or modules may at least temporarily be in data exchange with each other, e.g., via a network connection or respective interfaces. Consequently, individual units may be located apart from each other.

The interface unit may comprise an interface for data exchange with a local server or a central web server via internet connection for receiving the medial images.

The user interface may be adapted to interface with one or more users of the system, e.g., by displaying the result of the processing of the computing unit to the user (e.g., in a graphical user interface) or by allowing the user to make inputs for arriving at a medical diagnosis.

According to other aspects, one or more example embodiments of the present invention further relate to an integrated data management system (or healthcare information system) comprising the above system and an image archiving system configured to acquire, store and/or forward whole slide images. Thereby, the interface unit may be configured to receive the whole slide images from the image archiving system. According to some examples, the image archiving system may be realized as a cloud storage or as a local or spread storage, e.g., as a PACS (Picture Archiving and Communication System).

According to other aspects, the systems are adapted to implement the inventive method in their various aspects for providing a candidate medical finding. The advantages described in connection with the method aspects may also be realized by the correspondingly configured systems'components.

According to another aspect, the present invention is directed to a computer program product comprising program elements which induce a computing unit of systems herein described to perform the steps according to one or more of the above method aspects and examples as herein described, when the program elements are loaded into a memory of the computing unit.

According to another aspect, the present invention is directed to a computer-readable medium on which program elements are stored that are readable and executable by a computing unit of systems herein described to perform the steps according to one or more method aspects and examples as herein described, when the program elements are executed by the computing unit.

The realization of one or more example embodiments of the present invention by a non-transitory computer program product and/or a non-transitory computer-readable medium has the advantage that already existing providing systems can be easily adapted by software updates in order to work as proposed by one or more example embodiments of the present invention.

The computer program product can be, for example, a computer program or comprise another element next to the computer program as such. This other element can be hardware, e.g., a memory device, on which the computer program is stored, a hardware key for using the computer program and the like, and/or software, e.g., a documentation or a software key for using the computer program. The computer program product may further comprise development material, a runtime system and/or databases or libraries. The computer program product may be distributed among several computer instances.

Independent of the grammatical term usage, individuals with male, female or other gender identities are included within the term.

Characteristics, features and advantages of the above-described invention, as well as the manner they are achieved, become clearer and more understandable in the light of the following description of embodiments, which will be described in detail with respect to the figures. This following description does not limit the present invention on the contained embodiments. Same components, parts or steps can be labeled with the same reference signs in different figures. In general, the figures are not drawn to scale. In the following:

### BRIEF DESCRIPTION OF THE DRAWINGS

FIG. 1 schematically depicts a system for providing a conditional information and/or a representation of a whole slide image according to an embodiment,

FIG. 2 schematically depicts a method for providing a conditional information of a whole slide image according to an embodiment,

FIG. 3 schematically depicts data flows in a method for providing a conditional information of a whole slide image according to an embodiment,

FIG. 4 schematically depicts a method for providing a representation of a whole slide image according to an embodiment,

FIG. 5 schematically depicts data flows in a method for providing a representation of a whole slide image according to an embodiment,

FIG. 6 schematically depicts a graphical user interface for displaying a representation of a whole slide image according to an embodiment, and

FIG. 7 schematically depicts a trained function for determining a conditional information of a medical image according to an embodiment.

### DETAILED DESCRIPTION

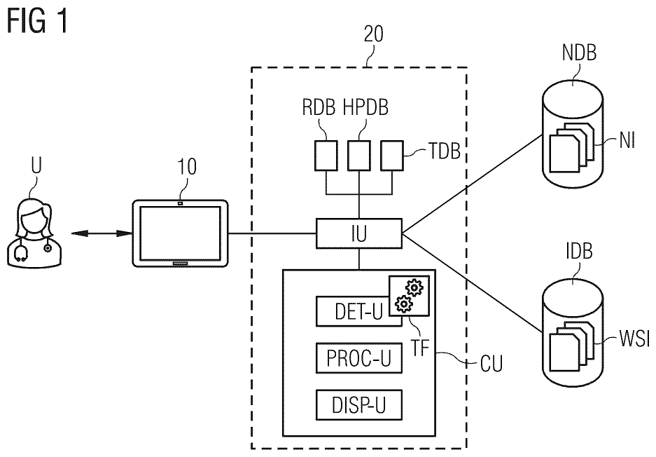

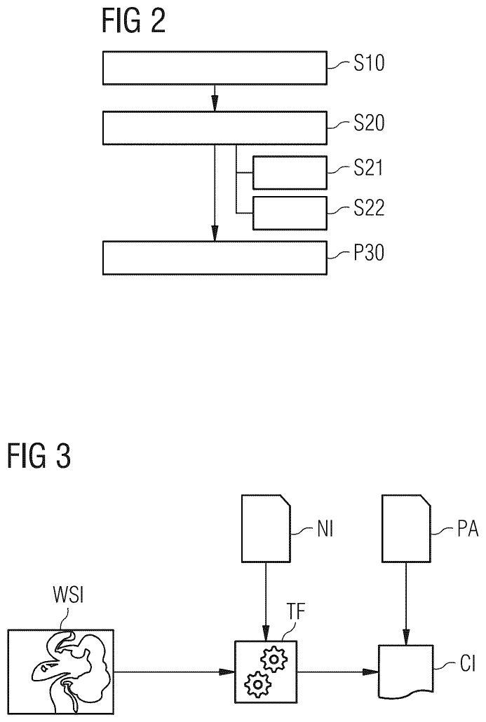

FIG. 1 depicts a healthcare information system 1 for providing a conditional information CI and/or a representation RE of a whole slide image WSI. In this regard, healthcare information system 1 is adapted to perform the methods according to one or more embodiments, e.g., as further described with reference to FIGS. 2 to 6.

A user U of healthcare information system 1, according to some examples, may generally relate to a healthcare professional such as a physician, clinician, technician, pathologist and so forth.

Healthcare information system 1 may comprises a user interface 10 and a processing system 20. Further, system 1 may comprise or be connected to a database DB generally configured for storing and/or forwarding whole slide images WSI and supplementary (non-image) information. The database DB may comprise one or more storage devices for whole slide images WSI (also denoted as image database) which may be realized in the form of one or more cloud storage, local or spread storage modules, e.g., as a PACS (Picture Archiving and Communication System).

According to some examples, the healthcare information system may comprise one or more medical imaging modalities (not shown) for acquiring whole slide images WSI, such as a slide scanning system.

In general, a whole slide image WSI depicts tissue slices prepared from tissue samples of a patient. The slices may be prepared by cutting tissue samples into micrometer-thick slices. The whole slide image WSI depicts the tissue slices in microscopic observation. Accordingly, a tissue slice can show the fine tissue structure of the tissue sample and in particular the cell structure or the cells contained in the tissue sample.

In general, a whole slide image will show different tissue types, such as healthy tissue with healthy cells, cancerous tissue with cancerous cells and others, such as amorphous tissue or necrotic tissue. In particular, the cancerous cells may indicate the tumor type and grade or how the tumor would respond to one or more therapy options. This is because the morphology of the cells and the cancerous regions provides indications as to how aggressive the tumor is and as to how susceptible it is vis-à-vis various treatment options. Besides, also the healthy cells may be relevant for the reviewing pathologist, as it may be derived from the morphology of healthy tissue how it would suffer under a particular treatment option and, thus, what would be the most likely side effects of the treatment option.

Whole slide images WSI may depict tissue stained with a particular histopathological stain in order to highlight features in the tissue slides for inspection. The most common stain is the H&E (hematoxylin and eosin) stain. Accordingly, the whole slide image WSI will generally be based on a H&E staining. Furthermore, also different stains may be considered, for instance, in the form of comparative whole slide images F-WSI of the patient. These comparative whole slide images F-WSI may be part of the supplementary information SI which may be retrieved for safeguarding a treatment response prediction TRP.

Whole slide images WSI may be formatted according to the DICOM format. DICOM (=Digital Imaging and Communications in Medicine) is an open standard for the communication and management of medical imaging information and related data in healthcare informatics. DICOM may be used for storing and transmitting medical images and associated information enabling the integration of medical imaging. A DICOM data object consists of a number of attributes, including items such as the patient's name, ID, etc., and also special attributes containing the image pixel data and metadata extracted from the image data. The metadata may be stored in the so-called DICOM header.

Non-image information NI may be any data providing additional information relating to the patient and/or the whole slide image WSI. Non-image image NI may relate to non-image examination results such as lab data, vital signs records (comprising, e.g., ECG data, blood pressure values, ventilation parameters, oxygen saturation levels) and so forth. It may comprise structured and unstructured medical text reports, referral letters, procedural records, etc. relating to prior examinations or the current examination of the patient. Further, non-image data may comprise personal information of the patient such as gender, age, weight, insurance details, and so forth.

The non-image information NI may be stored in the healthcare information system 40. For instance, the non-image information NI may be stored in dedicated databases of the healthcare information system 40 such as an electronic health/medical record database (not shown).

Of note, the term non-image information does not exclude that the data contains image data. According to some examples, the term non-image data may be construed as not comprising whole-slide images WSI as such. However, also the non-image information may comprise image data, e.g., in the form of image elements in medical reports or the like.

User interface 10 may comprise a display unit and an input unit. User interface 10 may be embodied by a mobile device such as a smartphone or tablet computer. Further, user interface 10 may be embodied as a workstation in the form of a desktop PC or laptop. The input unit may be integrated in the display unit, e.g., in the form of a touch screen. As an alternative or in addition to that, the input unit may comprise a keyboard, a mouse or a digital pen and any combination thereof. The display unit may be configured for displaying representations RE of the whole slide image WSI, medical report templates RT, image processing results FI, in a graphical user interface GUI, wherein all elements to be shown are arranged according to a displaying protocol HP.

User interface 10 may further comprise an interface computing unit configured to execute at least one software component for serving the display unit and the input unit in order to provide a graphical user interface for allowing the user to select a target patient's case to be reviewed and making various inputs. In addition, the interface computing unit may be configured to communicate with medical information system 40 or processing system 20 for receiving the whole slide images WSI and any non-image information NI. The user U may activate the software component via user interface 10 and may acquire the software component, e.g., by downloading it from an internet application store. According to an example, the software component may also be a client-server computer program in the form of a web application running in a web browser. The interface computing unit may be a general processor, central processing unit, control processor, graphics processing unit, digital signal processor, three-dimensional rendering processor, image processor, application specific integrated circuit, field programmable gate array, digital circuit, analog circuit, combinations thereof, or other now known devices for processing image data. User interface 10 may also be embodied as a client.

The processing system 20 may comprise a computing unit CU and an interface unit IU. Further, the processing system 20 may comprise or be connected to a plurality of dedicated repositories or databases including the image database IDB, the non-image database NDB, a reporting database RDB, a tool database TDB, and a displaying protocol database HPDB. According to some examples, the databases IDB, NDB, RDB, TDB, HPDB may be part of the healthcare information system 1.

The reporting database RDB is a storage device such a cloud or local storage serving as an archive for preconfigured reporting templates RT.

Thereby, a reporting template RT may be seen as a building block for a medical report. Reporting template RT may be configured for editing by the user via user interface 10. Reporting template RT may comprise one or more data fields into which diagnostic information specific for the patient and/or the underlying whole slide image WSI may be specified. The data fields may be empty fields or placeholders for various kinds of data such as text, measurement values or images.

A reporting template RT may be specific to a certain diagnostic task or use case (which may be indicated by the conditional information CI as herein described).

The displaying protocol database HPDB is a storage device such a cloud or local storage serving as an archive for preconfigured displaying protocols HP.

A displaying protocol HP may comprise a series of rules in the form of computer-executable instructions for optimally arranging medical information, in particular, medical images in a graphical user interface according to a dedicated use case r diagnostic task. A displaying protocol may set out which kind of representations of a set of whole slide images WSI, C-WSI and other information are to be produced and where these elements are to be shown in the graphical user interface GUI.

The tool database TDB is a storage device such as a cloud or a local storage serving as a repository for preconfigured image processing tools IPT.

Image processing tools IPT are generally configured to be applied to whole slide images WSI, C-WSI. In other words, these are tools which are configured to process image data in order to provide a corresponding processing result FI. The image processing result FI may be related to a medical finding. According to some examples, the processing tools IPT may be specialized for a certain use-case such as a type of histopathological staining (e.g., H&E staining or antibody staining) and/or a certain type of image processing result (e.g., tumor grading, classification of cancerous tissue, cell counts). For instance, one of the image processing tools IPT may be configured to predict a treatment response, e.g., based on a quantification of PD-L1 positive tumor cells, while another image processing tool IPT may be configured to measure microsatellite instabilities, e.g., based on MLH1 stained slides. Generally, the tool database TDB may comprise all image processing algorithms which are available for processing all kinds of image data which may occur at a certain healthcare facility or diagnostic workplace.

Computing unit CU may be a processor. The processor may be a general processor, central processing unit, control processor, graphics processing unit, digital signal processor, three-dimensional rendering processor, image processor, application specific integrated circuit, field programmable gate array, digital circuit, analog circuit, combinations thereof, or other now known device for processing image data. The processor may be single device or multiple devices operating in serial, parallel, or separately. The processor may be a main processor of a computer, such as a laptop or desktop computer, or may be a processor for handling some tasks in a larger system, such as in the medical information system or the server. The processor is configured by instructions, design, hardware, and/or software to perform the steps discussed herein. Further, computing unit CU may comprise a memory such as a RAM for temporally loading the whole slide images WSI and any intermediate processing results.

According to some examples, such memory may as well be comprised in user interface 10.

Computing unit CU may comprise sub-units DET-U, PROC-U, DISP-U configured to process whole slide images WSI and non-image information NI in order to provide a representation RE.

Sub-unit DET-U is configured to determine a conditional information CI for whole slide images WSI. The conditional information CI may comprise the circumstances under which the whole slide image WSI was acquired and/or how the whole slide image WSI was prepared.

This includes the tissue block and section from the block and the staining used. Sub-unit DET-U may be specifically configured to extract the conditional information CI directly from the image data of the whole slide image WSI, that is the pixels and voxels. Non-image information NI may be considered as an auxiliary source. To do so, sub-unit DET-U is configured to run an accordingly configured trained function TF which has been trained to derive the conditional information CI.

Sub-unit PROC-U is configured to use the conditional information CI for automating some of the steps required for deriving a medical diagnosis from the whole slide image WSI by a user U. This may involve providing an image processing result by selecting an image processing tool IPT from the tool database TDB according to the conditional information CI and applying it to the whole slide image WSI. Further, sub-unit PROC-U may be configured to generate one or more representations RE from the whole slide image WSI for displaying to the user U which fit the conditional information CI. Further, sub-unit PROC-U may use the image acquisition information to select suitable reporting templates RT and displaying protocols HP from the reporting database RDB or displaying protocol database HPDB.

Sub-unit DISP-U is a displaying module or unit. Specifically, sub-unit DISP-U may be configured to use the displaying protocol HP selected by the processing unit PROC-U and arrange any representations RE, image processing results FI, reporting templates RT, or any other data elements in a graphic user interface GUI according to the displaying protocol.

The designation of the distinct sub-units DET-U, PROC-U, DISP-U is to be construed by way of example and not as a limitation. Accordingly, sub-units DET-U, PROC-U, DISP-U may be integrated to form one single unit (e.g., in the form of “the computing unit”) or can be embodied by computer code segments configured to execute the corresponding method steps running on a processor or the like of processing system 20. The same holds true with respect to the interface computing unit. Each sub-unit DET-U, PROC-U, DISP-U and the interface computing unit may be individually connected to other sub-units and/or other components of the system 1 where data exchange is needed to perform the method steps.

Processing system 20 and the interface computing unit(s) together may constitute the computing unit CU of the system 1. Of note, the layout of this computing unit CU, i.e., the physical distribution of the interface computing unit and sub-units DET-U, PROC-U, DISP-U is, in principle, arbitrary. Specifically, processing system 20 may also be integrated in user interface 10. As already mentioned, processing system 20 may alternatively be embodied as a server system, e.g., a cloud server, or a local server, e.g., located on a hospital or radiology site. According to such implementation, user interface 10 could be designated as a “frontend” or “client” facing the user, while processing system 20 could then be conceived as a “backend” or server. Communication between user interface 10 and processing system 20 may be carried out using the https-protocol, for instance. The computational power of the system may be distributed between the server and the client (i.e., user interface 10). In a “thin client” system, the majority of the computational capabilities exists at the server. In a “thick client” system, more of the computational capabilities, and possibly data, exist on the client.

Individual components of system 1 may be at least temporarily connected to each other for data transfer and/or exchange. User interface 10 communicates with processing system 20 via interface unit IU to exchange, e.g., whole slide images WSI, elements of a graphical user interface GUI or any user input made. Further, processing system 20 may communicate interface unit IU with the database 40 and/or the dedicated database TDB, HPDB, RDB. The interface unit IU data exchange may be realized as hardware-or software-interface, e.g., a PCI-bus, USB or fire-wire. Data transfer may be realized using a network connection. The network may be realized as local area network (LAN), e.g., an intranet or a wide area network (WAN). Network connection is preferably wireless, e.g., as wireless LAN (WLAN or Wi-Fi). Further, the network may comprise a combination of different network examples.

FIG. 2 depicts a method for providing a conditional information CI according to an embodiment. Corresponding data streams are illustrated in FIG. 3. The method comprises several steps. The order of the steps does not necessarily correspond to the numbering of the steps but may also vary between different embodiments of the present invention. Further, individual steps or a sequence of steps may be repeated.

At step S10, a case identifier of the digital pathology case to be reviewed is obtained. The case identifier may be any suitable electronic identifier which identifies the patient and/or the task in the healthcare information system 1 such as a patient's name or case ID. The case identifier may be obtained based on a manual selection of the case by the user U, e.g., by selecting a case in the worklist in a graphical user interface GUI running in the user interface 10.

At step S20, a conditional information CI is determined. This may be done by querying the databases IDB, NDB connected to the processing system 20 using the case identifier. With that, available whole slide images WSI and non-image information NI may be accessed. The data thus obtained may be input in a trained function TF hosted in the processing system 20, which trained function TF is adapted to extract the conditional information CI from the whole slide images WSI and/or the non-image information NI.

Specifically, at optional sub-step S21, non-image information NI is obtained. According to some examples, the non-image information NI may relate to meta-data already comprised in the whole slide image WSI, e.g., in a header of the whole slide image WSI, in particular a DICOM header. In addition to that or as an alternative, non-image information NI may be obtained from additional data sources such as the electronic medical record of the patient. The non-image information NI may contain natural language text.

The non-image information NI may be searched for indications of the clinical circumstances of the case, such as suspected diagnoses, a clinical history of the patient, the diagnostic task, and the like. This may involve searching the non-image information NI for conditional information CI using a natural language processing function which may likewise be hosted in the computing unit CU. The information thus derived may be used to complement conditional information CI obtained from other sources.

At optional sub-step S22, the conditional information CI may be obtained from the image data comprised in the whole slide images WSI. To this end, the whole slide images WSI associated to the case by virtue of the case identifier may be analyzed. According to some examples, this may involve determining a staining of the whole slide images WSI. According to some examples this may be done by simply analyzing the color values of the whole slide images WSI (as stainings may feature distinct colors). According to other examples, a machine-learned function TF may be provided and applied to the whole slide images at step S22. The machine-learned function TF has been configured to recognize the type of the staining of whole slide images WSI. With that, more subtle differences between stainings may be exploited.

Specifically, at optional sub-step S23, the information derived from non-image information NI may be used to verify the conditional information CI obtained based on image data so as to combine the conditional information CI obtained from these two complementary sources. This may involve comparing the conditional information CI from the two sources. If, based on the comparison, discrepancies are detected, the conditional information CI may be corrected and/or the user may be notified.

Further, at optional sub-step S24, one or more prior actions PA of the user may be determined. The prior actions PA may be previous interactions of the user U with the user interface 10, in particular, for generating prior representations RE. The prior actions PA describe “viewing behavior” or user preference and may involve the selection of certain stainings, an order of selecting staining, a displaying order, regions of interest, zoom levels and other viewing parameters. The prior actions PA may be added to the conditional information CI.

At step P30, the conditional information CI is provided.

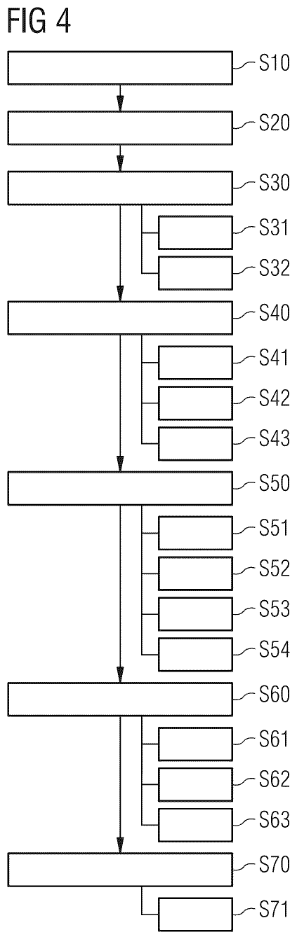

This may involve showing the conditional information CI in the user interface 10, e.g., in a suitable graphical user interface GUI. Moreover, step P30 may comprise providing the conditional information CI for subsequent image processing steps as shown in connection with FIGS. 4 to 6.

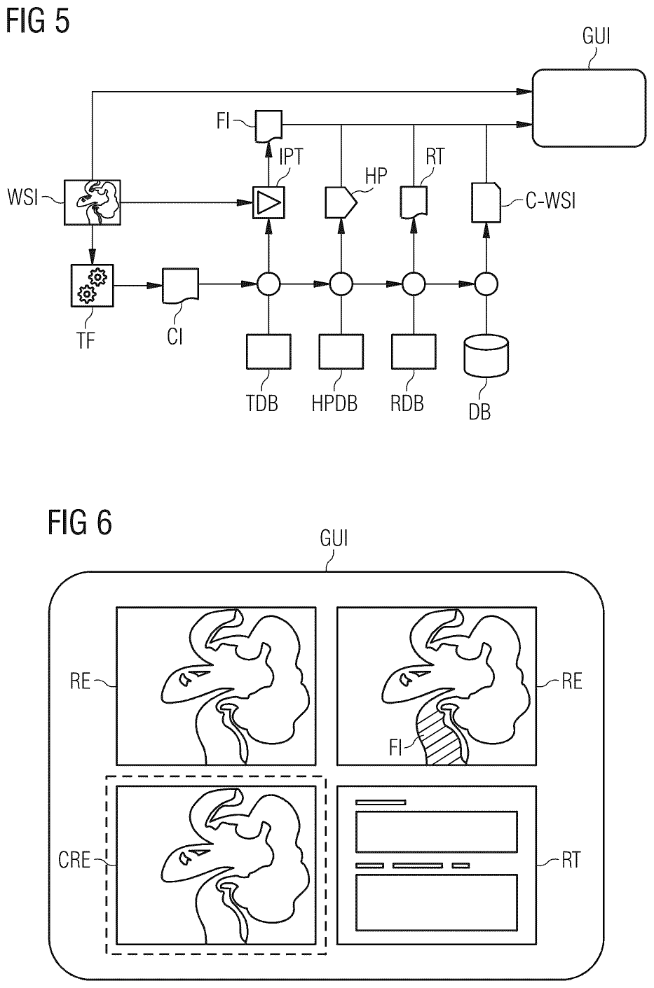

FIG. 4 depicts a method for displaying a representation RE of a whole slide image WSI according to an embodiment. Corresponding data streams are illustrated in FIG. 5. FIG. 6 shows a corresponding graphical user interface GUI for displaying the representation RE and further information in a reading and reporting workflow. The method comprises several steps. The order of the steps does not necessarily correspond to the numbering of the steps but may also vary between different embodiments of the present invention. Further, individual steps or a sequence of steps may be repeated. Steps S10 and S20 correspond to steps S10 and S20 of FIG. 3.

At step S30, the conditional information CI is used to generate one or more appropriate representations RE of the whole slide image WSI for displaying to a user U in the user interface 10. This may involve determining the type of representations RE coming into question for the conditional information CI and selecting and processing appropriate image data, in particular, excerpts, from the whole slide image WSI.

According to some examples, first, an appropriate whole slide image WSI may be selected from the whole slide images WSI associated to the case identifier. This may happen at optional step S31. Specifically, the conditional information CI may indicate a desired staining type. For instance, the prior actions PA may indicate that the particular user U tends to start his session with reviewing an H&E stained whole slide image. That followed, the whole slide images WSI may be searched for whole slide images WSI which have been stained with the desired staining type. From this subgroup, a further selection may be made, e.g., based on image quality or the tissue type displayed or the slice position of the whole slide image.

Further, step S30 may involve, at optional sub-step S32 determining appropriate display settings for the representation RE (optional sub-step I31). For instance, the display settings may comprise a contrast, brightness, zoom level, region of interest, image enhancement and the like.

The display settings may be preconfigured and assigned to certain diagnostic use cases. As the conditional information CI may indicate the diagnostic use case, it becomes possible to determine the displaying setting based on the conditional information CI.

At step S40, the representation(s) RE generated in step I30 are displayed to the user U in the user interface 10. This may comprise generating appropriate control signals for operating the user interface 10 to display the representation(s) RE.

Optionally, the representation(s) RE may be generated according to a displaying protocol HP. The displaying protocol HP may define what kind of representation(s) RE are to be displayed and where they are to be displayed in a graphical user interface GUI. Further, the displaying protocol HP may set out subsequent processing steps such as the retrieval of comparative whole slide images C-WSI or the application of image processing tools IPT. At step S41, a preconfigured displaying protocol HP may be retrieved from the displaying protocol database HPDB which matches the conditional information CI. To this end, a lookup operation may be performed in the displaying protocol database HPDB for a displaying protocol HP corresponding to the conditional information CI. Specifically, an association linking the displaying protocols HP in the displaying protocol database HPDB with conditional information CI or corresponding use-cases may be used to find suitable displaying protocols HP.

At optional step S50, a comparative whole slide image C-WSI may be retrieved and provided alongside the whole slide image WSI. A comparative whole slide image C-WSI may generally relate to a whole slide image which is helpful for arriving at a medical diagnosis based on the whole slide image WSI. As such, the comparative whole slide image C-WSI may relate to a prior study of the patient. As an alternative, the comparative whole slide image C-WSI may be a similar image of different patient which may already have been diagnosed. Further, the comparative whole slide image C-WSI may be from the same biopsy block as the whole slide image WSI. In particular, the comparative whole slide image C-WSI may have been prepared from an adjacent slice or section. Further, the comparative whole slide image C-WSI may be stained with a different histopathological staining than the whole slide image WSI. The type of the comparative whole slide image C-WSI may be defined in the displaying protocol HP or linked to the diagnostic use case respectively identified based on the conditional information CI.

Specifically, at optional sub-step S51, the comparative whole slide image C-WSI may be retrieved from the database DB. Thereby, medical images may be retrieved which match the conditional information CI. For instance, if the conditional information CI indicates that a certain staining is required, the database DB may be searched for images of the patient which have been stained with the required stain. Further conditions such as image quality and slice proximity with respect to the whole slide image WSI already presented may be considered. To make such selection, the whole slide images of a patient in the database coming into question may be subjected to the same processing as the whole slide image WSI for deriving a conditional information CI. In particular, the staining type may be determined using the machine-learned function.

At optional sub-step S52, the comparative whole slide image C-WSI may be subjected to an appropriate image processing for preparing a comparative representation CRE therefrom which can be readily compared to the representation RE of the whole slide image WSI. In particular the same image processing may be applied to the comparative medica image CI which was used for the whole slide image WSI. In particular, the same display settings may be used.

According to some examples, an image registration may be calculated between the whole slide image WSI and the comparative whole slide image C-WSI. The image registration may be conceived as a mathematical transformation from the coordinate system of the whole slide image WSI to the coordinate system comparative whole slide image C-WSI. Accordingly, spatially resolved image elements, such as findings, annotations, regions of interest may be mapped between the whole slide images. With that, the representation RE and the comparative representation CRE may zero in on the same region of interest.

At sub-step S54, the comparative representation CRE is displayed together with the representation RE. Specifically, the comparative representation CRE may be displayed according to the displaying protocol HP selected at step S41 in the graphical user interface GUI.

At optional step S60, an image processing result FI may be generated based on the whole slide image WSI and provided to the user U via the graphical user interface GUI. The image processing result FI may be generated according to the conditional information CI and/or the displaying protocol HP. The image processing result FI may relate to a medical finding, a measurement or a segmentation extracted from the whole slide image WSI. The image processing result FI may be generated using a corresponding image processing algorithm or tool IPT which may be executed by the computing unit.

Specifically, at sub-step S61, an image processing tool IPT may be selected from the tool database TDB according to the conditional information CI (or according to the diagnostic use case and/or displaying protocol HP respectively identified based on the conditional information CI). The look-up of the image processing tool IPT may be based on an association linking the image processing tools IPT in the tool database TDB with conditional information CI (or displaying protocols HP/diagnostic use cases).

At sub-step S62, the selected image processing tool IPT may be applied to the whole slide image WSI. Optionally, at sub-step S62, the image processing tool IPT may also be applied to any comparative whole slide image C-WSI to obtain a comparative image processing result FI.

At sub-step S63, the image processing result FI is displayed to the user U in the graphical user interface GUI. The displaying may be in accordance with any rules in the selected displaying protocol HP specifying the arrangement of image processing results FI in the graphical user interface GUI.

At optional step S70, a reporting template RT on the basis of which medical report may be completed by the user U may be selected and provided. The reporting template RT may be provided to the user U in the graphical user interface GUI. Thereby, the location of the reporting template RT in the GUI may be determined by the displaying protocol HP.

Specifically, at step S71, a reporting template RT is retrieved from the report database RDB which matches the conditional information CI. To this end, a lookup operation may be performed in the reporting database RDB for reporting template RT corresponding to the conditional information CI. Specifically, an association linking the reporting templates RT with displaying protocols HP or diagnostic use cases (both of which may be identified based on the conditional information CI) may be used to find correct reporting templates RT.

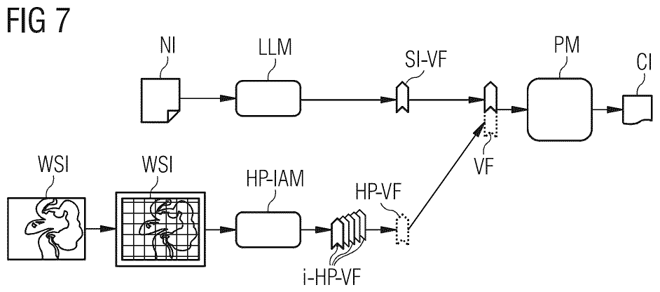

In FIG. 7, a schematic representation of a machine-learned function TF according to an embodiment is shown.

As shown in FIG. 7, the machine-learned function TF may comprise two branches: a branch for processing whole slide images WSI with a histopathology image analysis module HP-IAM, and a branch for processing non-image information NI (in particular, structured or unstructured text) with a data extraction module LLM.

The respective processing modules HP-IAM, LLM in the individual branches may be configured to extract observables from the corresponding input data which are relevant for predicting the conditional information CI relevant for the case. The observables may respectively have the form of a feature vector HP-VF, SI-VF. The feature vectors HP-VF, SI-VF may be learned by machine learning techniques and/or may be pre-defined. The latter may come into question where it is already clear that a certain information is particularly relevant, such as the staining.

As for the histopathology image analysis module HP-IAM, there are various implementations available for extracting feature vectors HP-VF from whole slide images WSI. For instance, it would be possible to employ a CNN. For instance, a ResNet, in particular a ResNet-18 or a ResNet-50 may be used to convert image data of whole slide images WSI into feature vectors HP-VF.

To enable efficient processing, each whole slide image WSI, may be divided into N tiles or patches. For each of the tiles, an intermediate feature vector i-HP-VF may be extracted which may be averaged to obtain the final feature vector HP-VF for the respective whole slide image WSI.

The data extraction module LLM may comprise a transformer architecture. In particular, the data extraction module LLM may comprise a large language module LLM. A transformer network is a neural network architecture generally comprising an encoder, a decoder or both an encoder and decoder. In some instances, the encoders and/or decoders are composed of several corresponding encoding layers and decoding layers, respectively. Within each encoding and decoding layer is an attention mechanism. The attention mechanism, sometimes called self-attention, relates data items (such as words) within a series of data items to other data items within the series. The self-attention mechanism for instance allows the model to examine a group of words within a sentence and determine the relative importance other groups of words within that sentence have to the word being examined.

The encoder, in particular, may be configured to transform the input (text) into a numerical representation. The numerical representation may comprise a vector per input token (e.g., per word). The encoder may be configured to implement an attention mechanism so that each vector of a token is affected by the other tokens in the input. In particular, the encoder may be configured such that the representations resolve the desired output of the transformer network.

The decoder, in particular, may be configured to transform an input into a sequence of output tokens. In particular, the decoder may be configured to implement a masked self-attention mechanism so that each vector of a token is affected only by the other tokens to one side of a sequence. Further, the decoder may be auto-regressive meaning in that intermediate results (such as a previously predicted sequence of tokens) are fed back.

According to some examples, the output of the encoder is input into the decoder. Further, the transformer network may comprise a classification module or unit configured to map the output of the encoder or decoder to a set of learned outputs such as the desired feature vector SI-VF.

Training of a transformer model according to some examples may happen in two stages, a pretraining and a fine-tuning stage. In the pretraining stage, a transformer model may be trained on a large corpus of data to learn the underlying semantics of the problem. Such pre-trained transformer models are available for different languages. For certain applications described herein, the fine-tuning may comprise further training the transformer network with medical texts with expert annotated meanings and/or medical ontologies. With the latter, in particular, the transformer model according to some examples may learn typical relations and synonyms of medical expressions.

For a review on transformer networks, reference is made to Vaswani et al., “Attention Is All You Need”, in arXiv: 1706.03762, Jun. 12, 2017, the contents of which are herein included by reference in their entirety.

According to some examples, data extraction module LLM is configured to derive a number of predefined features SI-VF from the electronic medical record EMR, which are to be added to the feature vector(s) extracted from the whole slide images WSI. The predefined features SI-VF may be predetermined influencing factors which would affect the decision making of a human user U regarding one or more viewing options, such as the diagnostic task, the available stainings, the location of the biopsy or resurrection, the affected organs, relevant lab values (such as PSA in case of prostate cancer), co-morbidities, already performed therapies and their success, metastasis, etc.

The individual feature vectors HP-VF, SI-VF may be concatenated to form a combined feature vector VF. The combined feature vector VF may then be input in a prediction module PM. For the prediction module PM, a regressor model can be used. The prediction module PM may output the prediction for conditional information CI for one or more viewing options, such as displaying protocols HP, image processing tools IPT, reporting templates RT and the like. The viewing options may be pre-configured, for instance, based on the use cases within an organization.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Wherever meaningful, individual embodiments or their individual aspects and features can be combined or exchanged with one another without limiting or widening the scope of the present invention. Advantages which are described with respect to one embodiment of the present invention are, wherever applicable, also advantageous to other embodiments of the present invention. Independent of the grammatical term usage, individuals with male, female or other gender identities are included within the term.

What is claimed is:

1. A computer-implemented method for providing a representation of a whole slide image, the computer-implemented method comprising:
obtaining a case identifier indicating a digital pathology case to be reviewed by a user on a user interface, in a corresponding diagnostic task of the user, wherein the digital pathology case is associated with at least one whole slide image stored in an image database;
determining conditional information applicable to the corresponding diagnostic task based on the case identifier;
generating a representation of the at least one whole slide image for display on the user interface by processing the at least one whole slide image according to the conditional information; and
providing the representation for display on the user interface.

2. The computer-implemented method according to claim 1, wherein determining the conditional information comprises:
obtaining non-image information associated with the case identifier from a database that is different from the image database; and
determining the conditional information based on the non-image information.

3. The computer-implemented method according to claim 1, wherein determining the conditional information comprises:
providing a machine-learned function configured to determine a conditional information based on image data included in whole slide images; and
applying the machine-learned function to the at least one whole slide image.

4. The computer-implemented method according to claim 3, wherein
the machine-learned function is configured to determine histopathological stainings of whole slide images, and
the conditional information includes a staining type of the at least one whole slide image.

5. The computer-implemented method according to claim 2, wherein determining the conditional information further comprises:
combining the conditional information based on the non-image information with conditional information based on image data to provide the conditional information.

6. The computer-implemented method according to claim 1, wherein
the digital pathology case is associated with a plurality of different whole slide images, and
generating the representation includes selecting the at least one whole slide image from the plurality of different whole slide images based on the conditional information.

7. The computer-implemented method according to claim 6, wherein
the plurality of different whole slide images are stained with different histopathological stainings, and
generating the representation includes
determining, based on the conditional information, a desired histopathological staining,
determining, among the plurality of different whole slide images, a subgroup stained with the desired histopathological staining, and
selecting the at least one whole slide image from the subgroup.

8. The computer-implemented method according to claim 1, wherein
generating the representation includes
determining a display setting for generating the representation based on the conditional information, and
processing the at least one whole slide image according to the display setting, and
the display setting includes one or more of a contrast setting, a brightness, an intensity windowing, an image enhancement, a look-up table, a viewing plane, a segmentation mask, a region of interest, or a zoom level.

9. The computer-implemented method according to claim 1, wherein
the conditional information includes an indication of a region of interest in the at least one whole slide image, and
generating the representation includes processing the at least one whole slide image to provide a view of the at least one whole slide image which is zoomed in on the region of interest.

10. The computer-implemented method according claim 1, further comprising:
retrieving, from the image database, a comparative whole slide image based on the conditional information,
processing the comparative whole slide image to generate a comparative representation for displaying on the user interface, and
displaying the comparative representation on the user interface.

11. The computer-implemented method according to claim 10, wherein generating the comparative representation comprises at least one of:
processing the comparative whole slide image in an analogous way to the at least one whole slide image, or
calculating an image registration between the at least one whole slide image and the comparative whole slide image and processing the comparative whole slide image using the image registration.

12. The computer-implemented method according to claim 10, wherein
the at least one whole slide image is prepared from a section of a biopsy block,
the conditional information includes an indication of a location of the section of the biopsy block, and
the comparative whole slide image is selected based on the location.

13. The computer-implemented method according to claim 1, further comprising:
selecting, based on the conditional information, a displaying protocol including a rule set for displaying one or more representations of whole slide images on a user interface, wherein
displaying the one or more representations includes displaying a representation based on the displaying protocol.

14. The computer-implemented method according to claim 1, further comprising:
selecting, based on the conditional information, an image processing tool from a plurality of image processing tools configured to provide an image processing result,
applying the image processing tool to the at least one whole slide image to generate the image processing result, and
displaying the image processing result on the user interface.

15. The computer-implemented method according to claim 1, further comprising:
monitoring one or more prior actions of the user; and
determining the conditional information based on the one or more prior actions.

16. A system for providing a representation of a whole slide image for displaying the representation on a user interface, the system comprising:
an interface unit in data communication with the user interface and an image database, the user interface being configured to display information to a user, and the image database being configured to store whole slide images; and
a computing unit configured to
obtain a case identifier indicating a digital pathology case to be reviewed by a user on the user interface within a corresponding diagnostic task, the digital pathology case being associated with at least one whole slide image stored in the image database,
determine conditional information applicable to the corresponding diagnostic task, based on the case identifier,
generate a representation of the at least one whole slide image for displaying on the user interface by processing the at least one whole slide image according to the conditional information, and
provide the representation to the user interface.

17. A non-transitory computer program product comprising program elements that induce a computing unit to perform the computer-implemented method according to claim 1 when the program elements are executed by the computing unit.

18. A non-transitory computer-readable medium storing program elements that, when executed by a computing unit, cause the computing unit to perform the computer-implemented method according to claim 1.

19. The computer-implemented method according to claim 12, wherein the comparative whole slide image is selected based on a proximity to the location.

20. The computer-implemented method according to claim 14, wherein the image processing result is displayed in the representation.
